(19) United States
(12) Patent Application Publication
(10) Pub. No.: US 2024/0316369 A1
(43) Pub. Date: Sep. 26, 2024

(54) WEARABLE DEVICE FOR GENERATING EXTRACORPOREAL SHOCK WAVES

(71) Applicant: CORTERY AB, Domsten (SE)

(72) Inventors: Filip Ludwig Peters, Domsten (SE); Christopher Lee Stokely, Houston, TX (US); Lars Gunnar Henrik Staaf, Lindome (SE); Mohammad Yusuf Mulla, NorrKöping (SE); Christer Johansson, Göteborg (SE)

(21) Appl. No.: 18/578,635

(22) PCT Filed: Jul. 12, 2022
(86) PCT No.: PCT/EP2022/069521

§ 371 Date: Jan. 11, 2024

(30) Foreign Application Priority Data
Jul. 12, 2021 (EP) 21185026.8

(51) Int. Cl. A61N 7/02; A61N 7/00

(52) U.S. Cl. CPC A61N 7/02; A61N 2007/0004; A61N 2007/0052; A61N 2007/0078; A61N 2007/0082

(57) ABSTRACT

A wearable device for generating extracorporeal shock waves in a thoracic region of a user includes a shock wave transducer unit to generate extracorporeal shock waves and configured to be placed on the skin of the user to apply shock wave therap. At least one proximity sensor measures the proximity of the shock wave transducer unit relative to the user's skin. A positioning mechanism is configured to controllably position the shock wave transducer unit. A processor is configured to transmit information to the shock wave transducer unit for generating extracorporeal shock waves.

### CROSS-REFERENCE TO RELATED APPLICATIONS

This application is the U.S. National Stage of PCT/EP2022/069521 filed on Jul. 12, 2022, which claims priority to European Patent Application 21185026.8 filed on Jul. 12, 2021, the entire content of both are incorporated herein by reference in their entirety.

### FIELD OF INVENTION

The present disclosure relates to measurement of cardiac function for assessing cardiovascular health and generation of ultrasonic shock waves for therapeutic use. The present disclosure relates more particularly, but not limited to, a wearable device for generating extracorporeal shock waves in a thoracic region of a user.

### BACKGROUND OF THE INVENTION

Cardiovascular disease is a leading cause of death and disability worldwide. Although many pharmacological and device-based therapies have been developed for cardiovascular diseases such as resistant hypertension and myocardial infarction, many still have disappointing clinical outcomes. Use of ultrasound for cardiovascular diagnosis has been applied widely and may evolve similarly into a strategy for cardiovascular therapy because of its noninvasive and nonionizing characteristics. Ultrasound therapy has been used as a resource for device-based treatment in neurosurgery, cancers, and cardiology, alone and/or in combination with other interventions.

However, existing systems and methods are costly and error-prone due to the sticking force of the sensor/probe. This specification recognizes that there is a need for a portable and efficient device to obtain and measure cardiac function and generate extracorporeal shock waves, without requiring a user and/or assistant to have any specific abilities or training.

Each human has different heart motion, respiratory movement, and blood circulation that can reduce the target accuracy and acoustic energy accumulation of ultrasound therapies. Interference in acoustic pathways may reduce the required energy and induce non-target injury, preventing achievement of the therapeutic goal. High intensity focused ultrasound delivery to the human thoracic region may lead to undesired lesions and decrease the intensity of the energy that arrives at the targets. Lower intensity ultrasound over longer sessions and/or periods of time may provide a safer way of administering ultrasound therapy, thereby minimizing unwanted side-effects. However, patients suffering from cardiovascular disease often have a plurality of comorbidities and precipitating factors that affect their health. Hence, adaptive and/or personalized treatments are required in order to be effective. Treating multiple cardiac conditions at the same time further requires a device that can continuously adapt to new information about the user's cardiac health and adapt therapies accordingly.

Ultrasound therapy typically requires a visit to a healthcare professional and clinical environment. Patients are often more susceptible to hospital-acquired infections and keeping patients out of the hospital can reduce the risk of acquiring diseases. A reduced need for hospital-based visits may further reduce the amount of hospital resources needed to treat a patient.

Thus, in view of the above, there is a long-felt need in the healthcare industry to address the aforementioned deficiencies and inadequacies.

### SUMMARY OF THE INVENTION

A purpose of the present disclosure is to provide a wearable device for assessing a cardiac function of the heart of a user and generating extracorporeal shock waves for treating of heart diseases of the heart and vessels around and on the heart.

A further purpose of the present disclosure is to provide a personalized ultrasound therapy to each patient depending on their body features and/or disease characteristics, thereby minimizing adverse effects and helping to reduce unwanted side-effects.

Generally, low-frequency ultrasound has good penetration that can reach deeper targets and initiate predominantly mechanical effects on cell membranes with negligible temperature increase (<0.01° C.), thereby possibly depolarizing membranes to activate voltage-gated sodium channels and voltage-gated calcium channels and to influence cells' excitability. However, high-frequency ultrasound has a shorter wavelength and better spatial resolution than low-frequency ultrasound. The high-frequency ultrasound is centrally deposited, which is helpful in imaging. Fast attenuation of the high-frequency ultrasound may cause thermal loss and poor penetration when applied to the delivery of skin treatment.

It is commonly known that ultrasound can induce a wide range of bio-effects in soft tissues. An advantage of the presently disclosed approach is the ability to generate controlled biological effects non-invasively. Depending on which biological tissue reaction is sought, the magnitude and frequency of exposure parameters can be adjusted. A further advantage of the presently disclosed approach is that the adaptive ultrasound pulsing system can be used for cost-efficiently personalizing cardiovascular ultrasound treatments.

The present disclosure relates to a device for generating extracorporeal shock waves in a thoracic region of a user, the preferred embodiment is a wearable device. The device is preferably configured to receive cardiac health information. The device may comprise at least one cardiac sensor, such as an ultrasound sensor, e.g. an ultrasound receiver, for non-invasively acquiring cardiac data, and/or be configured to receive information from an invasive cardiac sensor, e.g. a pacemaker. A shock wave transducer unit or shock wave transducer units can be provided in order to generate extracorporeal shock waves. Via control of the device the shock wave transducer unit(s) can be placed on the skin of the user to apply shock wave therapy, e.g. in the form of ultrasound therapy.

The shock wave transducer units can be arranged in an array of shock wave transducer units.

The array of shock wave transducer units can function as the cardiac sensor for gaining cardiac health information of the heart of the user. Since the array of shock wave transducer units can function as the cardiac sensor, the cardiac sensor can gain the cardiac health information about different parts of the heart without having to move the wearable device. The array of shock wave transducer units can be a planar array and/or a linear array for the phase array transducer units.

The wearable device may further comprise at least one proximity sensor. The proximity sensor can measure the proximity of the shock wave transducer unit relative to the user's skin. The measured proximity is the distance between the shock wave transducer unit and the skin of the user and will determine how tight the fit of the transducer is to the user's skin and thus the real distance between the shock wave transducer unit and the heart or different parts of the heart. The wearable device may further comprise a positioning mechanism configured to controllably position the shock wave transducer unit and the at least one cardiac sensor relative to the skin of the user. The at least one cardiac sensor is preferably part of the shock wave transducer unit.

The wearable device may comprise a processor configured to transmit information to the shock wave transducer unit for generating extracorporeal shock waves. The information can comprise information on therapy parameters including but not limited to information on location, frequency, spatial average temporal average, duty cycle and/or duration of a shock wave therapy.

A pressure sensor is an example of a proximity sensor, which can be configured to measure the proximity of a shock wave transducer to the user's skin. This implies that a sensor which can detect the contact state between the shock wave transducer unit and the user's skin can be used. The pressure sensors may be any instruments or devices that transform the magnitude of a physical pressure being exerted onto the sensor into an output signal that can be used to establish a quantitative value for the pressure.

Advantageously, the shock wave transducer unit is configured to generate extracorporeal shock waves and is configured to be placed on the skin of the user, preferably with a sticking force chosen to optimize a shock wave therapy. The sticking force is the contact force between the shock wave transducer unit and the skin of the user. Advantageously, said contact force can be predefined and measured by means of the proximity sensor. Based on a predefined value an optimized shock wave therapy routine can be provided.

Shock wave therapy may be taken to mean any therapeutic ultrasound modality including piezoelectric crystals configured to be electrically stimulated and release high frequency sound waves to propagate through tissue, with part of the generated energy being absorbed and another part being reflected by fluid, cells, and/or connective tissue. Ultrasound therapy can be adjusted according to clinical application and tissue characteristics using different parameters (including but not limited to frequency, amplitude and/or pulse duration) and may be optimized to maximize absorption to enable therapeutic applications. The presently disclosed device is preferably configured to execute a plurality of functions and can thereby provide a long-term in-home therapy, without requiring specialist training.

The wearable device may comprise an array of shock wave transducer units. The array of shock wave transducer units can be controlled independently so that the generated shock wave from the array can be controlled regarding direction and focus. That enables that a location of the wearable device on an area in the thoracic region of the user will suffice and the wearable device can treat the heart and any part of the heart of the user without having to be moved.

The positioning mechanism may be configured to position the shock wave transducer unit within an area in the thoracic region of the user. The positioning mechanism can improve the control of the position of the shock wave transducer on the skin of the user. As a result, the shock wave therapy can be applied in a guided manner. In an embodiment, the positioning mechanism comprises a guiding channel. A guiding channel can be provided in a bottom surface of the device, which faces the skin of the user during shock wave therapy. Guiding channels can guide the shock wave transducer unit in a plane defined by said bottom surface of the device towards the skin of the user. Advantageously, the shock wave transducer unit can be guided through the guiding channels on a planar surface, improving the guidance and the positioning control of the shock wave transducer unit. The bottom surface of the device is the surface of the device configured to face the skin of the user.

The presently disclosed device may be equipped with a plurality of sensors providing sensor data, e.g. cardiac data.

Continuous positioning of the shock wave transducer unit and acquisition of sensor data can be automatized and coupled with an artificial intelligence based approach such as a Case-Based Expert System, and/or implement a fuzzy logic control system.

A personalized shock wave therapy parameter can be calculated by a machine learning model. Consequently, the device can be configured to continuously identify, and scan regions of the heart that needs to be treated and apply region-based shock waves.

The shock waves from the shock wave transducer unit(s) may have an energy level of 0.02 mJ/mm² or above.

### BRIEF DESCRIPTION OF THE DRAWINGS

The invention will in the following be described in greater detail with reference to the accompanying drawings:

FIG. 1 shows a block diagram of the connections between the various components of the wearable device for generating extracorporeal shock waves in a thoracic region of a user, in accordance with one embodiment of the present disclosure;

FIG. 2 shows a bottom view of the wearable device, in accordance with one embodiment of the present disclosure;

FIG. 3 shows a side view of the wearable device, in accordance with one embodiment of the present disclosure;

FIG. 4 shows a monitoring application installed within the handheld computing device, in accordance with one embodiment of the present disclosure;

FIG. 5 shows a perspective view of the handheld computing device placed against the body or chest of a user, in accordance with at least one embodiment;

FIG. 6 shows a perspective view of interactions between the ultrasonic sensor and the heart of the user, in accordance with at least one embodiment;

FIG. 7 shows a first exploded view of the placement of the wearable device against the chest of the user, in accordance with at least one embodiment;

FIG. 8 shows a second exploded view of the placement of the wearable device against the chest of the user, in accordance with at least one embodiment;

FIG. 9 shows a third exploded view of the placement of the wearable device against the chest of the user, in accordance with at least one embodiment;

FIG. 10 shows a perspective view of placing the wearable device into a pocket of a vest, in accordance with at least one embodiment; and

FIG. 11 shows a perspective view of a matrix or array of ultrasound transducers, in accordance with at least one embodiment.

### DETAILED DESCRIPTION OF THE INVENTION

From one aspect, the present disclosure relates to a device comprising a mobile device comprising a processor and a memory and being adapted to be configured as described herein.

The present disclosure further relates to a wearable device configured to perform a set of instructions which when executed by a computing device cause the computing device to generate extracorporeal shock waves in a thoracic region of a user.

In an embodiment, the wearable device comprises a computing device. The computing device can be a mobile computing device, which can be handheld, e.g. a smartphone. Thus, in an embodiment, the wearable device comprises a handheld computing device. In a further embodiment, the wearable device comprises a display screen. Preferably, the handheld computing device may have a display screen.

In an embodiment, the wearable device is configured to control a smartphone of the user, so that the power mode of the phone is adjusted to a minimized generation of electronic RF noise so that influence from and interruption by the phone on the correct function of the piezoelectric transducers is/are eliminated or at least reduced.

Accordingly, one advantage of the present disclosure is that the wearable device can use both processing and power of the handheld computing device, such as a smartphone, for example to conduct Doppler shift based measurement, and by using a cheap and existing technology of carbon fiber together with lateral stickers and automated sensor positioning to guarantee the precise positioning of the ultrasonic sensor and avoid any external pressure on the blood vessels that may deform the vessel shape or diameter leading to errors. Hence, from one aspect the wearable device may act as a gadget for the computing device.

In an embodiment, the presently disclosed device comprises a circuit board, such as a PCB, for connecting the electronic components of the device, e.g. the shock wave transducer unit, the sensors, the positioning mechanism and optionally the processor. The PCB can refer to a printed wiring board, printed wiring card, or a printed circuit board (PCB) that mechanically supports and electrically connects electrical or electronic components of the presently disclosed device using conductive tracks, pads, and other features etched from one or more sheet layers of copper laminated onto and/or between sheet layers of a non-conductive substrate. The interfacing circuit board (PCB) can be connected to the handheld computing device mentioned above.

The device comprises at least one data processor for executing program components for executing user-or system-generated requests. The processor may comprise specialized processing units such as integrated system (bus) controllers, memory management control units, floating point units, graphics processing units, digital signal processing units, etc. Processor may further comprise a microprocessor, such as AMD® ATHLON® microprocessor, DURON® microprocessor OR OPTERON® microprocessor, ARM's application, embedded or secure processors, IBM® POWERPC®, INTEL′S CORE® processor, ITANIUM® processor, XEON® processor, CELERON® processor or other line of processors, etc. Processor may be implemented using a mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), AlazarTech controller systems, etc.

The processor may be disposed of in communication with one or more input/output (I/O) devices via an I/O interface. I/O interface may employ communication protocols/methods such as, without limitation, audio, analog, digital, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), etc.

In an embodiment, the PCB is connected to one or more of the following: an analog-to-digital converter (ADC) for converting analog ultrasonic data into digital data, a micro-controlling unit with a power and data transmission port, one or more large bandwidth operational amplifiers circuits, a plurality of digital buffers, at least two signal mixers for precise Doppler calculation, a plurality of filters suitable for an operating range of a piezoelectric ultrasonic sensor, a plurality of bidirectional drivers for a micro linear actuator and a servo motor, a plurality of headers and a plurality of PWM lines to provide the power to the micro linear actuator and the servo motor.

In an embodiment, the PCB is connected to a handheld computing device via a cable and/or interface with data and power lines, wherein the cable/interface receives power from the handheld computing device. In a further embodiment, the wearable device receives power through a cable from an external electric power system.

In an embodiment, the wearable device comprises a boost circuit for providing power feed to the shock wave transducer unit. In a further embodiment, the boost circuit comprises a low equivalent series resistance (ESR) capacitor and utilizes an accumulated charge on high capacitance. The accumulated charge can be acquired from the handheld computing device via the power and data transmission cable during idle time.

In an embodiment, the device further comprises a battery. Thus, the wearable device can receive power from the external battery that can supply electrical power to the PCB. The battery can be based on Lithium Polymer (Li-Poly) and Lithium-Ion (Li-lon). Furthermore, the battery can be operated by a power management integrated circuit such as power MOSFETs. Alternatively, the wearable device can be powered by the handheld computing device.

In one embodiment, the wearable device can be powered by a power source, where the power source can be one or batteries, AC mains, an inductive power transfer with no physical contact, where the inductive power transfer is powered by e.g. AC mains, fiber optic power supply, where the fiber optic power supply can e.g. comprise an optical-to-electrical converter, like a solar cell, for providing electrical energy, a solar array for providing electrical energy, a wind-up manual system for providing electrical energy e.g. by a electrical generator driven by the wind-up manual system, or an energy-harvesting system for providing electrical energy from a magnetic field. In the embodiment where inductive power transfer relies upon an AC main, the wearable device may comprise a filter to shunt power transients of high frequency and/or large magnitude.

A great advantage of employing handheld computing device technologies is to provide a cheap and reliable access to cardiac function measurement, and add the feature of an extracorporeal shock wave to harness the thermal and non-thermal effects of high-intensity focused ultrasound (HIFU) and extracorporeal shock waves to treat heart failure, for relief and mobility restoration. The wearable device can work with automated precision sensor positioning, and negligible error of the sensor weight.

In an embodiment, the device further comprises a memory and/or a server for providing instructions. The memory may be a non-volatile memory or a volatile memory. Examples of non-volatile memory may include, but are not limited to flash memory, a Read Only Memory (ROM), a Programmable duty cycle le ROM (PROM), Erasable PROM (EPROM), and Electrically EPROM (EEPROM) memory. Examples of volatile memory may include but are not limited to Dynamic Random-Access Memory (DRAM), and Static Random-Access memory (SRAM). Alternatively, the device can comprise a processor and a memory and can be adapted to perform plurality of events.

Cardiac health information as used herein may include cardiac sensor data and/or self-reported cardiac health data. Said cardiac sensor data may include but is not limited to non-invasive cardiac sensor data such as electrocardiogra photoplethysmography (PPG), ultrasound, phonocardiography (PCG), myocardial perfusion by thallium scintigraphy and/or data pertaining to ejection fraction. Said cardiac sensor data may furthermore include invasive cardiac sensor data from sensors such as implantable intracardiac pressure sensors, pacemakers defibrillators and/or any other implantable cardiac devices. Self-reported cardiac health data may include but is not limited to one or more self-reported symptoms questionnaire(s) pertaining to angina, Canadian Cardiovascular Society score, degree of heart disease medicine use, self-administered 6-min walk test and/or any physical symptoms arising from an exercise test.

Pressure sensors as used herein may be, but are not limited, to potentiometric pressure sensors, inductive pressure sensor, capacitive pressure sensors, piezoelectric pressure sensors, strain gauge pressure sensors, variable reluctance pressure sensors, aneroid barometer sensors, Manometer Sensors, Bourdon Tube Pressure Sensors, Vacuum Pressure Sensors, Sealed Pressure Sensors.

As stated previously the cardiac sensor may be an ultrasonic sensor for measuring a cardiac function of the heart of the user. The ultrasonic sensor can for example be a piezoelectric ultrasonic sensor. The ultrasonic sensor may comprise a MEMS ultrasonic sensor.

The cardiac sensor may be an electronic stethoscope. Said electronic stethoscope may be any device configured to electronically amplify the body sounds by means of acoustic conversion sound waves obtained through a chest piece into electrical signals, which can then be amplified for optimal listening. An electronic stethoscope may include but is not limited to Micro-Electro-Mechanical System (MEMS) microphones, electret condenser microphones (ECM), accelerophone and/or piezoelectric microphones.

The device may further comprises one or more sensors selected from the group of: photoplethysmography (PPG) sensors, electrocardiogramsors, inertial measurement unit (IMU) sensors, e.g. such that the device is configured to determine whether the device has been correctly positioned. These may also be part of the cardiac sensor.

The shock wave transducer unit can comprise an ultrasonic transceiver to generate the extracorporeal shock waves. However, in some embodiments, the extracorporeal shock waves may be infrasound frequency shock waves.

In an embodiment, the presently disclosed device is configured to receive analog data from the user.

The proximity sensor ensures that the device can receive information regarding the predetermined proximity of the shock wave transducer unit to the user's skin and/or the pressure applied on the user's skin. Consequently, an error resulting from pressing the skin with the shock wave transducer unit can be minimized.

In an embodiment, the wearable device is configured to be individually calibrated to the user.

The presently disclosed device can comprise one or more rows of shock wave transducers, such as ultrasound transducers. Said transducers can be activated in accordance with the region of the heart that is to be analyzed and/or treated. Said transducers can be adjusted electronically and/or mechanically to ensure a good fit with the user's skin. The shock wave transducer unit may further comprise an electrohydraulic source, piezoelectric source, electromagnetic source with flat coil and/or electromagnetic source with cylindrical coil. Furthermore, the shock wave transducer unit may be configured as a signal generator mounted on a concave transducer to enable focus of ultrasound at one or more fixed focus lengths. Said configuration may be used to emit or generate beams to converge energy onto a target tissue, at a pre-determined depth from a tissue surface, with a minimal resolution and minimal effect on tissue between a surface and a target. Said configuration may enable its use in harnessing the thermal effects of ultrasound.

The present disclosure describes deploying extracorporeal shock wave treatment, wherein the user receives the shock wave therapy instructions which include disease type that needs to be treated, e.g. coronary artery disease and/or heart muscle stiffness. These instructions may be predefined rules/instructions based on the user's characteristics, e.g., body size, age, type, and/or severity of heart disease—e.g., in ischemic heart disease one may want to achieve angiogenic effects that may be achieved by using shock wave therapy parameters.

In an embodiment, the wearable device of any of the preceding claims, wherein the transducer units can be controllable independently for controlling the direction and/or the focal length of the generated extracorporeal shock waves.

The transducer units can be the shock wave transducer units.

The individual transducer units can be phase shifted with respect to each other. Depending on the relative phase shift between the transducer units, the position of the maximum of the total superposition wave at a plane with respect to the 1D or 2D array, can be positioned by adjusting the phases for each of the transducer units in the array. Using this method the shockwave can be steered to a specific angle (and position) without using any moving mechanical parts. Likewise, the ultrasound waves from the transducer units can be focused at a certain area of the heart, and the shockwave can be controlled regarding angle and focus on the heart.

In an embodiment, the array can be one-dimensional or two-dimensional.

If the array is one-dimensional, the generated extracorporeal shock waves can be controlled in one dimension. If the array is two-dimensional, the generated extracorporeal shock waves can be controlled in two dimensions.

In an embodiment, the at least one array of shock wave transducer units can be configured to generate electric signals as responses to reflected ultrasound waves from the heart.

That the transducer units can be two-way transducers means that the array of shock wave transducer units can generate in addition to the extracorporeal shock waves for treating the heart also generate ultrasound waves and register the reflected ultrasound waves for generating a picture of the heart and even a moving picture of the heart. In that way the heart can be (further) diagnosed and the diagnosis can be used to determine:
a. where on the heart of the patient the array of transducers should be focused,
b. how much power should be used for the shockwave,
c. how focused the shockwave should be,
d. how large the focal area on the heart should be,
e. whether the shockwave should be continuous or pulsed, and if pulsed possibly the frequency of the pulse and/or the duration of each pulse, etc.

The same shock wave transducer units can generate the extracorporeal shock waves for treating the heart and the ultrasound waves for imaging the heart. The difference between the waves is the energy density or intensity of the waves, where the shock waves have a higher intensity than the ultrasound waves.

When the reflected ultrasound waves enter the array of shock wave transducer units the reflected ultrasound waves create voltage/current responses from the shock wave transducer units depending on the strength of the amplitude of the vibrations/pressure wave. This means that the array of shock wave transducer units can be used as an active sensor array for monitoring the status of the heart. Since the reflected ultrasound waves will reach the different transducer units at different times, the array of shock wave transducer units can register the phase difference and build a picture of the heart based on the reflected ultrasound waves. The array of shock wave transducer units can be used to localize the heart in the extrapolated actuator/sensor array plane by using the amplitude and time delay between each sensor/actuator in the array.

In general, a controller sends electrical signals for controlling the transducer units, where the electrical signals are converted to ultrasound waves. Phased array systems are composed of an array of transducer units in 1D or 2D arrays and can transmit waves independently at different times or phase changes between the transducer units. To focus or to steer the ultrasound waves, time delays or phase changes are applied to the transducer units in order to create constructive interference of the wave fronts of the ultrasound wave from each transducer unit. Due to this effect, the ultrasound wave can be steered to a certain angle and/or the energy can be focused on any position of the heart.

After transmitting the ultrasound waves, the transducer units receive the reflected ultrasound waves from the heart as an echo, as well. By having the transducer units to be used inversely, the transducer units can convert the received reflected ultrasound waves into electrical signals that can be registered by the controller. In the same way the array of transducer units can steer and focus the generated ultrasound waves as mentioned above, the array of transducer units can determine the direction and origin of the reflected ultrasound waves. All received waves are converted to electrical signals and can be evaluated by signal analysis to obtain health status of the heart and/or the effectiveness of the transmitted shock wave to the heart.

The advantages of phased array systems like the array of transducer units include the ability to perform scanning of the ultrasound waves, which reduces inspection times by eliminating or reducing the need of mechanically moving the array of transducer units.

When the array of transducer units is used for generating an image of the heart the energy level of the generated ultrasound waves may be lower or much lower than 0.02 mJ/mm².

In an embodiment, the wearable device can comprise an acoustic transducer, like a microphone or an accelerophone, for detecting sound waves below 20 kHz or below 15 kHz, or the wearable device can be configured to detect cavitation using the shock wave transducer units.

The use of ultrasound in a fluid (like water) under certain physical conditions can result in cavitation, which can cause major collateral tissue damage. If cavitation is created at the heart tissue the cavitation must immediately be stopped so that the heart tissue is not damaged. Cavitation has a distinct human-audible sound that will indicate the presence of cavitation with a frequency range from 5 kHz to 20 kHz. The cavitation will sound like a collection of popping sounds. It is a distinct and unique sound.

Cavitation can be detected as broadband popping sounds above about 5 kHz. By signal processing, e.g. by Fourier transformation or any other transformation, the time window of the measure acoustics is transformed to a frequency window, and by comparing e.g. the amplitude at one frequency a little above 5 kHz or at least within the frequency range of the cavitation with the amplitude at another frequency outside the frequency range of the cavitation (e.g. below 5 kHz or above 20 kHz). The amplitude of the frequency within the frequency range of the cavitation will be relatively high when cavitation occurs compared to the amplitude of the frequency within the frequency range of the cavitation. When such high amplitudes are detected, the array of shock wave transducer units is switched off.

The acoustic transducer can be tailor-made to only be sensitive to the frequency range of the cavitation. When the acoustic transducer registers noise, that will be a sign of cavitation, the array of shock wave transducer units is switched off.

Since the ultrasound waves have a frequency above 20 kHz, the acoustic transducer, like a microphone or an accelerophone, only sensitive to sound waves below 20 KHz or below 15 kHz will not be able to register the ultrasound. In principle, the only noise will be the beat from the heart. Any registered peak between the heartbeat can be an indication of a cavitation and the array of shock wave transducer units is then switched off.

The frequency range of the cavitation may vary from person to person and/or from one array of shock wave transducer units to another array of shock wave transducer units, so that the acoustic transducer may be adapted to function optimally. That would be a simple design feature.

Cavitation generates an easily recognizable noise that a machine learning model can be trained to register, by providing different noises and which ones are cavitation and which ones are not as input. When the cavitation is registered the array of shock wave transducer units are switched off or the intensity or amplitude of the generated shock waves is reduced.

The acoustic transducer can preferably be sensitive within the range of 3-20 KHz, preferably within the range of 4-20 kHz, and most preferably within the range of 5-20 KHz.

The shock wave transducer units can be sensitive to frequencies within the range of 3-20 kHz, 4-20 kHz, or 5-20 kHz, so that the shock wave transducer units can register the cavitation like the above-mentioned acoustic transducer. When the cavitation is detected by the shock wave transducer units the shock wave transducer units will stop generating ultrasound shockwaves to save and avoid damaging the heart tissue.

In an embodiment, the wearable device can be configured to emit extracorporeal shockwaves to induce a regenerative cardiac therapy. Regenerative cardiac therapy induced through extracorporeal shockwaves has the benefit of being non-invasive, safe, non-cancerogenic and in the wearable device embodiment described herein, less resource intensive than existing modalities of administering this form of therapy.

Regenerative cardiac therapy may include but is not limited to therapy that positively affects heart function in ischaemia by induction of angiogenesis and postnatal vasculogenesis.

In an embodiment, the wearable device can comprise one or more cardiac rhythm detection sensors.

Cardiac rhythm detection sensors may comprise but is not limited to electrocardiogramatoplethysmography (PPG), magnetocardiogarphy (MCG), seismocardiography (SCG), phonocradiography (PCG) and/or echocardiography sensors. These cardiac rhythm detection sensors can be used to enable cardiac gating and/or detect adverse cardiac events of the patient. Examples of adverse events that can be detected through cardiac rhythm detection sensors may include but is not limited to heart arrhythmias, acute coronary syndromes, acute heart failure, pulmonary health conditions, embolisms, strokes, etc.

Cardiac gating protocols that may be used include but are not limited to prospective high-pitch dual-source protocols, prospective step and shoot protocols, retrospective gated helical protocols. An common example of cardiac gating includes targeting the cardiac shockwave therapy under a ECG-based R-wave gating protocol.

The cardiac rhythm detection sensor(s) can be the shock wave transducer units or one or more external cardiac rhythm detection sensor(s) listening to the heartbeat.

If the shock wave transducer units is supposed to stimulate the heart, it may be advantageous to stimulate the heart at the same position in the heartbeat cycle. That will e.g. be advantageous when treating myocardial ischemia by targeting the area surrounding the left ventricle. In such a scenario, one may want to stimulate the myocardium on a cellular level to start a regenerative program on a cellular level. Controlling the administration of this therapy in the same position allows for more effective therapy to be administered and less leakage of shockwave energy to unwanted areas of the heart. This principle can be applied to a number of cardiac shockwave therapy modalities, including but not limited to treating heart arrhythmias, heart failure and cardiomyopathy.

In one embodiment, the wearable device can comprise one or more breath detection sensors.

The breath detection sensor(s) can be the shock wave transducer units listening to the sound caused by the breathing going into and out of the lungs.

The breath detection sensors can be used to enable the shockwave therapy to be administered under breath gating. Breath-gating can allow for the shockwave therapy to be administered in a more controlled manner, for example when the patient is breathing less heavily and/or breathing out and/or when there is less patient movement that can negatively impact the.

Said breath detection sensors may include but are not limited to electrocardiogramatoplethysmography (PPG), magnetocardiogarphy (MCG), seismocardiography (SCG), phonocradiography (PCG), infrared camera and/or echocardiography sensors.

A breath detection model can be applied to the data from said breath detection sensors to identify episodes of breathing and non-breathing. Said breath-detection model can be a classification model that has been trained on a number of annotated patient data points, based on episodes of breathing and/non-breathing.

For treating some diseases, e.g. diseases aimed at regenerative therapy of the myocardium such as coronary artery syndrome, it is advantageous to synchronize the shock waves with the breathing of the user. Since the chest will move during breathing cycle, the distance between the wearable device and the heart may change during the breathing cycle. By generating the shock waves at the same time in the breathing cycle, the focus of the shock waves will be at the right spot of the heart, making the effect of the therapy as localized and effective as possible.

The array of shock wave transducer units can be used to create an image of the heart, and the distance between the shock wave transducer units and the heart can be determined as a function of the position in the breathing cycle. With that knowledge and using the breath detection sensor(s) the focus of the shock wave transducer units can be varied during the breathing cycle so that the focus of the shock wave transducer units is always at the correct position of the heart.

### Ultrasound

One example of extracorporeal shock waves is ultrasound. Generally, ultrasound is defined as either low intensity or high intensity by determining whether the energy is below or above 1 W/cm². Further, the low-frequency and high-frequency ultrasound are classified by determining whether the frequency is below or above 1 MHz. The low-frequency ultrasound has good penetration that can reach deeper targets and initiate predominantly mechanical effects on cell membranes with negligible temperature increase (<0.01° C.), thereby depolarizing membranes to activate voltage-gated sodium channels and voltage-gated calcium channels and to influence cells' excitability. However, high-frequency ultrasound has a shorter wavelength and better spatial resolution than low-frequency ultrasound. The high-frequency ultrasound is centrally deposited, which is helpful in imaging. Fast attenuation of the high-frequency ultrasound may cause thermal loss and poor penetration when applied to the delivery of skin treatment.

A study compared the soundwave properties of bovine liver and heart muscle; the velocity, impedance, and density of heart muscle tissues are lower than those of the liver in the frequency range from 20 to 40 MHz. Further, loosely organized structures, such as a thrombus or an atheroma, lacking the normal collagen and elastin fiber support, can be destroyed easily by ultrasound, but vascular walls contain a thick collagen and elastin matrix, so they tolerate ultrasound of higher intensity and lower frequency. These features are the foundation of sonothrombolysis.

The present disclosure describes deploying ultrasound treatment, wherein the user receives the ultrasound therapy instructions which include disease type that needs to be treated, e.g. coronary artery disease and/or heart muscle stiffness. These instructions may be predefined rules/instructions based on the user's characteristics, e.g., body size, age, type, and/or severity of heart disease—e.g., in ischemic heart disease one may want to achieve angiogenic effects that may be achieved by using ultrasound therapy parameters set at a frequency of 1/1875 MHz; 15/25 mW/cm² spatial average temporal average (SATA); 20% duty cycle; 20 min/day).

In another example, one may want to achieve anti-inflammatory effects using parameters set at or close to a frequency of 1.5/3 MHz; 30/200 mW/cm² SATA; 20% duty cycle; 15/20 min/day). In a further example, one may want to achieve anti-degenerative effects using parameters set at or close to a frequency of 1 MHz; 50/110 mW/cm² SATA; 20%/50% duty cycle; 10/15 min/day). In a further example, one may want to achieve regenerative effects using parameters set at or close to a frequency of 1.5/1.6 MHz; 30/50/90 mW/cm² SATA; 20% duty cycle; 20 min/day). In a further example, one may want to achieve differentiation effects using parameters set at or close to a frequency of 1.5/1.6 MHz; 30/50/90 mW/cm² SATA; 20% duty cycle; 20 min/day).

The present disclosure also describes the semi-automated method of deploying ultrasound treatment. Furthermore, the present disclosure describes an automated method of deploying ultrasound treatment, where for the first time usage of an automatic calibration of the wearable device to map the location and size of the user's heart, the user has to perform various steps. Additionally, for the first time usage of manual calibration of the wearable device to map the location and size of the user's heart, the user has to perform various steps.

The automatic calibration of the wearable device, preferably including the location and parameters of the shock wave therapy can be determined through a mapping process using simultaneous acquisition of sensor data from two or more sensors, where the sensor data has been collected from the user. The intensity of a certain cardiac signal of interest can be compared between the two or more sensors. This can subsequently be visualised in the form of a heat map. A certain cardiac region of interest can be mapped out based on the location of the sensor that is receiving the strongest cardiac signal. Given knowledge of the patient, e.g. gender and size, the size and location of different regions of the heart can be approximated from physiological principles of the human body (e.g. average dimensions of the heart for a certain patient group). Based on the desired region of treatment, the shock wave therapy can be directed to the appropriate region of the heart.

If e.g. the location of the patient's aorta has been well determined, and the desired region of therapy is the left ventricle, then the shock wave therapy for an adult male can preferably be administered based on coordinates that are 5 centimetres away from the user's located aorta region (downwards when the patient is standing up) and 3 centimetres right relative to the location of the patient's located aorta as seen by a person facing the patient.

The cardiac data can come from sensor data based on a representative patient group. E.g. sensor data that is known to be representative of a certain cardiac region.

Cardiac data can be any sensor data emanating from the thoracic region of a patient.

Sensor data may comprise but is not limited to first sensor data from electrocardiogramography, seismocardiography, vibrational cardiography, phonocardiography, photoacoustics, cardiac imaging modalities (including but not limited to echocardiography, piezocardiography, CT-scans, MRI, SPECT-imaging, PET-scanning, etc.). The corresponding sensors for measuring the first sensor data are known within the field.

In one embodiment, the wearable device can be calibrated manually, whereby a healthcare professional determines the position of the shock wave transducer to optimize therapy for the patient. The optimal position of the shock wave transducer may be informed based on one or more imaging modalities, including but not limited to data from electrocardiogramatoplethysmography, seismocardiography, vibrational cardiography, phonocardiography, photoacoustics, cardiac imaging modalities (including but not limited to echocardiography, piezocardiography, CT-scans, MRI, SPECT-imaging, PET-scanning, etc.), to ensure that the shockwave therapy is administered in the optimal region of the patient. The optimal position of the shockwave transducer can be determined by assessing the size of the patient and ensuring a tight fit to enable that shock waves can be transmitted through the skin of the patient. This ensures that every time the patient puts on the wearable device, that the location of the shock wave transducers will be in the same region. The position mechanism of the shock wave transducer can in this case be fixed until a next manual and/or automatic calibration of the wearable device occurs.

### Positioning Mechanism

The presently disclosed device can be configured such that the shock wave transducer unit and/or the cardiac sensor(s) can move controllably by means of a positioning mechanism, such that the shock wave transducer unit, e.g. with an ultrasound sensor, can move across different regions of the user's thoracic region.

The positioning mechanism may comprise guiding channels, defining a predefined movement path, for guiding the movement of the shock wave transducer unit. One advantage thereof is that the channels allow the shockwave transducer unit to move via an automated controller that searches for the optimum signal through the movement path.

In an embodiment, the positioning mechanism can comprise a motor for engaging with the shock wave transducer unit, and possibly a pulley engaging with the motor. In a preferred embodiment, the device can be configured such that the motor and the pulley move controllably to position the shock wave transducer unit.

In an embodiment, the positioning mechanism comprises a micro linear actuator, which may comprise a driving side and a driven side for engaging with a motor from the driving side, and for engaging with the shock wave transducer unit from the driven side.

Additionally, the positioning mechanism may comprise a spring-based mechanism for engaging with the shock wave transducer unit.

In an embodiment, the wearable device is configured such that the positioning mechanism provides Pulse Width Modulation (PWM) control of the shock wave transducer unit. An average delivered power to the device can be controlled via PWM. PWM can drive the motor in on and off modes, which can be acquired by a micro-linear actuator.

### Shock Wave Therapy

The device can be configured to execute a plurality of events. The wearable device can for example be configured to (utilizing the cardiac sensor(s)) scan the user's heart, identify one or more regions of the user's heart and possibly detect one or more heart diseases. The wearable device may further be configured to detect a plurality of acoustic properties of a cardiovascular and extracorporeal tissue to optimize ultrasound therapy. Hence, the wearable device can be configured to determine an effective ultrasound therapy for the user in one or more regions of the user's thoracic region based on acoustic properties and/or demographic characteristics. The wearable device can for example be configured to move the shock wave transducer unit to the one or more identified regions and execute the ultrasound therapy in said one or more regions. Alternatively, the ultrasound therapy can be administered in the optimal location and with the optimal parameters for shock wave therapy.

In an embodiment, the presently disclosed device is configured to analyse a cardiac function of the heart of the user and to determine a location on the chest of the user where the shock wave transducer unit is positioned based on cardiac sensor data acquired non-invasively.

The cardiac function data can be collected for determining a cardiac function of the user. If the patient is known to suffer from e.g. coronary artery disease, an imaging modality such as ET ECG Exercise test, CPET cardiopulmonary exercise test, DSE dobutamine stress echocardiography, PET positron emission tomography, SPECT single photon emission computed tomography can be used to achieve the relevant cardiac function data. In addition to these monitoring methods, observational questionnaires and patient characteristics such as CCS (Canadian Cardiovascular Society Angina Class) angina class, Nitroglycerine consumption (expressed as number of tablets per day), NYHA (New York Heart Association) class and Seattle angina questionnaire can be used, to determine the cardiac function of the heart of the user. If the patient is known to suffer from e.g. heart failure with preserved ejection fraction (HFpEF) one use modalities such as echocardiography to assess the ejection fraction and/or blood sample testing such as NT-proBNP and/or BNP to assess the cardiac function of that patient. Similarly, for patients suffering from cardiomyopathy and indication of heart muscle inflammation may be of most interest.

To be able to determine the optimal location on the chest of the user where the shock wave transducer unit should be positioned and the shockwave parameters that should be transmitted, pre-defined treatment protocols from Tohoku University of Japan with respect to the shockwave output and the number of shots implemented to each spot and the protocol developed by the University of Essen, Germany, can be used.

As described herein, the device can be configured to create a map of the user's heart, wherein the map is stored in the memory and later be used to more efficiently position the cardiac sensor(s), and/or the shock wave transducer unit.

In an embodiment, the wearable device is configured to scan the heart by moving the shock wave transducer unit, e.g. comprising the cardiac sensor(s), across different regions of the user's thoracic region and collect data therefrom.

In an embodiment, the wearable device is configured to scan the heart by collecting data using the shock wave transducer unit, e.g. comprising the cardiac sensor(s), across different regions of the user's thoracic region and collect data therefrom.

The step of scanning the heart can mean collecting cardiac function data from different regions of the user's thoracic region, wherein the cardiac function data may include but is not limited to the first sensor data and the techniques for measuring the first sensor data.

In an embodiment, the wearable device is configured to compare the cardiac health of the user in the one or more regions to determine an efficacy of the ultrasound therapy over time. The wearable device can then be configured to update the ultrasound therapy based on the observed efficacy of the ultrasound therapy over time.

In an embodiment, the wearable device is configured to create a map of the user's heart, wherein the map is stored, e.g. in a memory of the device.

In an embodiment, the extracorporeal shock wave transducer unit is configured to harness non-thermal properties of ultrasound therapy.

In an embodiment, the wearable device is configured to collect information on the user's health condition through a questionnaire and/or patient health database.

The present disclosure relates to the existing manual method of deploying ultrasound treatment. For example, the user can receive the ultrasound therapy information, comprising disease type that needs to be treated, e.g. coronary artery disease and/or heart muscle stiffness. Then the user can position the shock wave generator or the hereby disclosed device to the therapy region (therapy region can be approximated from the user's body size and demographics. E.g., if it's a small person, the user may be given a small wearable structure (e.g. a vest) to hold the wearable device in place) based on ultrasound therapy instructions. A wearable structure and/or device may include any device that can be worn by a user over longer periods of time, can be placed on a user's thoracic region either by the user themselves and/or able to be held in place by a user for a period of time lasting more than 30 seconds. Lastly, the user can apply (administer) the shock wave therapy based on the instructions. These instructions may be predefined rules/instructions based on the user's characteristics, e.g., body size, age, type, and/or severity of heart disease. In a further embodiment, the user can manually adjust the pressure of the shockwave transducer unit to the skin of the user to optimize contact with the user's skin for optimal ultrasound penetration. Said pressure adjustment can be performed using a pressure adjustment knob which moves the shockwave transducer unit up and down. A pressure adjustment knob may in some cases be understood to be similarly constructed to a coarse adjustment knob of conventional microscopes that have been commonly used in clinical research.

The present disclosure further relates to a device configured to provide a semi-automated ultrasound treatment. Firstly, the user can receive ultrasound therapy information such as disease type that needs to be treated and therapy region, e.g. coronary artery disease and/or heart muscle stiffness. The user can position the device to the therapy region using previously mapped out heart regions from the first-time use calibration process. Then the device can provide the ultrasonic data and/or electronic stethoscope data (and/or other non-invasive cardiac data of the user) from the therapy region to the user. The user can therefore analyse severity of the disease. The risk analysis may be assessed by a risk assessment machine learning model and/or measurement of cardiac function such as ejection fraction and/or a patient self-reported questionnaire, etc). Then the user can identify the ultrasound therapy parameters such as intensity, duration, and/or pulsation frequency in one or more regions based on the user's characteristics, e.g., body size, age, type, and/or severity of heart disease (heart muscle stiffness may require an ultrasound therapy to relax the muscle and a coronary artery disease may require an ultrasound therapy to get rid off plaque). Lastly, the user can administer the shock wave therapy based on the identified therapy needs.

The ultrasound therapy information can be gained from e.g. a patient using ET ECG Exercise test, CPET cardiopulmonary exercise test, DSE dobutamine stress echocardiography, PET positron emission tomography, or SPECT single photon emission computed tomography. In addition to these monitoring methods, observational questionnaires and patient characteristics such as CCS (Canadian Cardiovascular Society Angina Class) angina class, Nitroglycerine consumption (expressed as number of tablets per day), NYHA (New York Heart Association) class or Seattle angina questionnaire can be used. The ultrasound therapy information will provide information about the heart of the patient.

The gained ultrasound therapy information can be used to determine the disease or disease type of the heart of the patient. The gained ultrasound therapy information about the disease or disease type of the heart of the patient can provide information about
a. where on the heart of the patient the array of transducers should be focused,
b. how much power should be used for the shockwave,
c. how focused the shockwave should be,
d. how large the focal area on the heart should be, and
e. whether the shockwave should be continuous or pulsed, and if pulsed possibly the frequency of the pulse and/or the duration of each pulse.

If the disease type e.g. turns out to be end-stage coronary artery disease, a treatment of three sessions per week for three weeks with up to 1200 impulses to the patient per session applied to the basal, middle, and apical segments of the left ventricle and not more than 100 impulses applied to one spot with an energy flux of 0.09 mJ/mm² will reduce the ischemia burden.

If the disease type e.g. turns out to be chronic ischemic heart failure preferably 300 impulses can be delivered to the ischemic area with an energy flux density of 0.38 mJ/mm² at a frequency of 4 Hz.

If the disease type e.g. turns out to be calcified aortic valve leaflets the hardened masses can be destructed by lithotripsy using two shockwaves at 100 KHz and at 3 MHz, respectively, with a time interval of 6 s. The combination of the different frequencies will disrupt the calcium deposits in the aortic valve cusps, avoiding thermal injury.

The gained ultrasound therapy information can instead of the disease or disease type of the heart of the patient provide information directly about
a. where on the heart of the patient the array of transducers should be focused,
b. how much power should be used for the shockwave,
c. how focused the shockwave should be,
d. how large the focal area on the heart should be, and
e. whether the shockwave should be continuous or pulsed, and if pulsed possibly the frequency of the pulse and/or the duration of each pulse.

The present disclosure further relates to a device configured to provide an automated ultrasound treatment. The user can have access to the sensor data, such as ultrasonic data and/or electronic stethoscope data (and/or other non-invasive cardiac data of the user) from a plurality of regions of the user. The user can therefore identify the one or more diseases and can analyse the severity of said one or more diseases in one or more regions. This can be done by employing a disease type and severity machine learning model, e.g., classification model trained on gold standards such as calcification index, plaque buildup in coronary arteries, measurement of cardiac function such as ejection fraction, a patient self-reported health outcome/wellbeing questionnaire, etc. Then the user can identify the shock wave therapy parameters (intensity, duration, and/or pulsation frequency) in one or more regions based on the user's characteristics, e.g. body size, age, type, and/or severity of heart disease (heart muscle stiffness may require an ultrasound therapy to relax the muscle and a coronary artery disease may require an ultrasound therapy to shoot away plaque). Lastly, the user can administer the shock wave therapy based on identified therapy needs.

For the first time usage of the automatic calibration of the wearable device to map the location and size of the user's heart, the user typically performs various steps. The user can access data of ultrasound sensor and/or electronic stethoscope from a plurality of regions (the data collection regions can be randomized and/or pre-set). Further, the user can identify the unique markers and/or patterns of each region for example by training a machine learning model to identify each region. Preferably, clustering methods can be used to segment data into different groups/regions. Furthermore, the position data corresponding to positioning of the sensors and/or shock wave transducer unit for each region can be stored in memory and/or cloud so that data can be accessed at a later point.

For the first time usage of manual calibration of the wearable device to map the location and size of the user's heart, the user typically perform various steps. Based on user characteristics (e.g., gender and body size), the device can be navigated to the desired region by a pre-set formula/decision rule. For example, if the user is female, 55 kg, and 70 years of age-position the shock wave transducer unit in the bottom right quadrant of the device when the disease to treat is mitral regurgitation.

The presently disclosed device can be configured to harness non-thermal properties of ultrasound therapy to generate stem cell differentiation, angiogenesis and anti-inflammatory effects, as a treatment for a plurality of diseases including but not limited to ischemic heart disease and/or fibrosis. Said non-thermal properties may be achieved through increased pressure and/or amplitude to generate microstreaming (whereby increased fluid movements can promote endothelial shear stress), jetting (whereby vascular permeability can be increased), bubble expansion and/or compression (whereby vascular permeability can be increased).

Specifically, the wearable device can be configured to harness thermal effects of ultrasound through increased pulse length and/or power applied by means of the shock wave transducer unit such that local tissue temperature, which may lead to liquefactive necrosis, can be increased.

Additionally, the wearable device can be configured to harness molecular effects. Said molecular effects may include but are not limited to the upregulation of angiogenic factors, increased nitric oxide synthase activity, anti-inflammatory properties, increased differentiation of myocytes, endothelial cells, and/or vascular smooth muscle cells.

The pulsation frequency and strength of the shock wave transducer may be aimed at suppressing hypertrophic cardiomyopathy and/or myocardial interstitial fibrosis.

The shock wave therapy may be aimed at enabling cardiac pacing. Alternatively, the presently disclosed device may be used to non-invasively reduce hypertension by affecting the nerves that control blood pressure. Furthermore, low-intensity ultrasound pulsations may be used to create anti-inflammatory effects. Ultrasound pulsations may also be configured to generate anti-inflammatory effects to target systemic microvascular inflammation.

Low-intensity ultrasound pulsations may be used to enhance angiogenesis to reduce left ventricular dysfunction. Additionally, low-intensity ultrasound pulsations may be used to enhance angiogenesis to ameliorate myocardial infarction.

The presently disclosed device can be used to apply ultrasound to liquefy blood clots, either independently or in combination with bubbles and anti-clotting agents, possibly being used to restore blood flow to regions of the brain affected by stroke and/or treating arterial thrombosis and/or deep vein thrombosis.

Furthermore, the presently disclosed device may be focused on increasing myocardial blood flow in ischemic myocardium and cardiac endothelial cells. Ultrasound has direct effects on tissue that are cardioprotective which may arise from increased tissue blood flow induced by ultrasound and/or metabolites released from endothelial cells which may offer cardio protection by increasing blood flow.

The presently disclosed device may also be focused on harnessing non-thermal properties of ultrasound therapy to generate stem cell differentiation, angiogenesis and anti-inflammatory effects, as a treatment for a plurality of diseases including but not limited to ischemic heart disease and/or fibrosis.

In some examples, the presently disclosed device is focused on harnessing non-thermal properties of ultrasound therapy to generate anti-inflammatory effects to inhibit fibroblast proliferation.

The presently disclosed device may be focused on harnessing non-thermal properties of ultrasound therapy to generate stem cell differentiation, angiogenesis and anti-inflammatory effects, as a treatment for a plurality of diseases including but not limited to pulmonary fibrosis, chronic obstructive pulmonary disease (COPD), respiratory syndromes and/or pulmonary embolism.

In an embodiment, the presently disclosed device is focused on harnessing thermal properties of ultrasound therapy to target and destroy tumorous cells.

In an embodiment, the presently disclosed device is focused on harnessing ultrasound therapy to target and destroy thrombus in a plurality of body regions of the user including but not limited to the lower limb region.

In an embodiment, the presently disclosed device is used to identify and/or treat deep vein thrombosis.

The presently disclosed device may be applied to one or more cardiovascular disease areas including but not limited to: Arteriovenous Malformations (AVM's), Atherosclerosis, Atrial Fibrillation, Cardiac Pacing, Cardiac Hypertrophy, Coarctation of the Aorta, Congestive Heart Failure, Deep Vein Thrombosis (DVT), Heart Valve Calcifications, Hematoma Management, Hypertension, Hypoplastic Left Heart Syndrome, Mitral Regurgitation, Peripheral Artery Disease, Septal Perforation, Varicose Veins, Ventricular Tachycardia, and Fibrillation and Heart Failure with Preserved Ejection Fraction (HFpEF).

### Machine Learning Model

A machine learning model underlying a machine learning system can be stored in a memory of device and/or the handheld computing device. The machine learning model can be trained by an adaptive clinical setting. The machine learning system can be integrated with the handheld computing device. Furthermore, the machine learning system can be executed remotely from a secondary handheld computing device. Additionally, the machine learning system can propose a recommended treatment, which can be accessed by the patient's clinician. The clinician can confirm the one or more recommended therapies including but not limited to the location, intensity, and/or frequency of the therapy.

In an embodiment, the machine learning system generates personalized ultrasound therapy for the user. For each user (patient), the machine learning system can measure the progression of the disease by comparing disease severity data in the period “A” with the disease severity data in the period “B”. Based on a dataset that includes all patients and/or all patients from multiple periods, the machine learning system can train a machine learning model to correlate x-variables (patient data and ultrasound therapy specifications, etc.) with the y-variables (disease progression). Based on the trained machine learning model, ultrasound therapy specifications (while holding other x-variables such as patient characteristics constant) are adjusted to minimize predicted disease progression (or in other words, maximize the efficacy of treatment). The machine learning system may include decision tree-based machine learning models, artificial neural networks, convolutional neural networks, logistic regression, naive Bayes, nearest neighbour, support vector machines, boosted tree learning methods, and/or generative neural networks.

The presently disclosed device may be configured to receive bio-sample data of the user. In operation, the user can receive his/her bio-sample from salivary glands in a form of saliva. Then the user can place the bio-sample on a reactant material having one or more reactant properties pertaining to chemical information of the user's body. Thereafter, the user can capture an image of the reactant material upon placing the bio-sample to obtain the bio-sample data. The device can then be configured to process the bio-sample data which acts as one of the decision points for generating personalized shock wave therapy. In an alternative embodiment, the device is configured to generate personalized shock wave therapy based on the medicine data of the user.

The device can be configured to utilize a machine learning model underlying a machine learning system stored in the memory of the user's handheld computing device. The machine learning model can for example be trained by an adaptive clinical setting. Furthermore, the machine learning system can be integrated with the handheld computing device. Additionally, the handheld computing device can also collect information about the user's health condition through a questionnaire and/or other patient health database.

A second handheld computing device may be connected to the wearable device, enabling remote control of the wearable device. The machine learning system can be executed remotely from a secondary handheld computing device. Furthermore, an external computing device, such as the second handheld computing device can receive data representing ultrasonic sensor data, which is recorded as a video file.

Machine learning essentially can be represented as a target variable T of data that should be mathematically approximated as accurately as possible based on an unknown mathematical combination of input variables called response variables A, B, C, D, E, . . . where T=f(A, B, C, D, E, . . . ), and the function f is not known a priori. This is similar to using a data fitting program but fitting the functional form of the equation to be fit has to be known. The function f is referred to as the machine learning model and the function f should be determined by careful selection of different algorithmic recipes based on the problem at hand, or by testing a large variety of different machine learning algorithms and ranking their accuracy for each class of algorithm. Machine learning should find models with as small a data requirement as possible; generate models that can correctly extrapolate or infer on new data scenarios; models should be simple to analyze, refit, and reuse; and the structure of the model should give insight into the problem. Moreover, the model should be able to provide suggestions on how to influence the input variables to adjust the target variable T in a controllable way. The adjustable input variables are referred to as levers-things that can be influenced or changed.

### Casing

In an embodiment, the wearable device further comprises a housing for accommodating at least a part of the shock wave transducer unit, the proximity sensor, the positioning mechanism, the cardiac sensor(s) and optionally the processor, wherein the housing is made of carbon fiber material. Advantageously, measurement error caused by the weight of the device can be minimized.

In a further embodiment, the wearable device can comprise a plurality of fixation pads at the bottom surface of the wearable device for allowing the wearable device to be attached to the user's skin.

In an advantageous embodiment, the wearable device comprises a container having acoustic impedance matching material or acoustic impedance matching liquid such as ultrasound gel. The housing can be designed with an opening in its side for a small sheet of ultrasound gel to be entered. The amount of acoustic impedance matching material or acoustic impedance matching liquid may be just enough for one-time use. Preferably, the acoustic impedance matching material or acoustic impedance matching liquid can be optimized for a certain intensity and/or duration of ultrasound therapy. Furthermore, the device can be provided with a brush and cleaning detergent to clean the acoustic impedance matching material or acoustic impedance matching liquid away from the transducer.

Additionally, the device can comprise a thin square sheet that connects to the shock wave transducer unit. Said thin square sheet may be configured for one-time use and may be connected by glue or attach mechanically with the transducers, where the acoustic impedance matching material or acoustic impedance matching liquid may be on the bottom of the sheet. Said thin square sheet may be configured to be removed after use.

Said acoustic impedance matching material or acoustic impedance matching liquid may comprise ultrasound gel to optimize the transmission of shockwaves into the tissue of the user. In an embodiment, said acoustic impedance matching gel further comprises an adhesive material that facilitates a tight fit between the shockwave transducers and the user's skin.

In a further embodiment, the device is configured to prompt the user to evenly apply acoustic impedance matching liquid across the thoracic region before ultrasound therapy commences.

The wearable device can be attachable to a vest. Said vest can be configured to ensure a good fit with the user's chest. Said vest may come in different sizes to ensure good fit across different patient populations. This can facilitate the wearable device being applied in the region of a user across different user populations. Said vest can be adjusted to increase pressure and/or improve contact of sensors and/or transducers with the patient's skin. Hence, such a vest can be part of a kit comprising the presently disclosed vest and the presently disclosed device.

### Detailed Description of the Drawings

The present description is best understood with reference to the figures and description set forth herein. Various embodiments of the present system and method have been discussed with reference to the figures. However, those skilled in the art will readily appreciate that the detailed description provided herein with respect to the figures are merely for explanatory purposes, as the present system and method may extend beyond the described embodiments. For instance, the teachings presented and the needs of a particular application may yield multiple alternative and suitable approaches to implement the functionality of any detail of the present systems and methods described herein. Therefore, any approach to implement the present system and method may extend beyond certain implementation choices in the following embodiments.

According to an embodiment herein, the methods of the present disclosure may be implemented by performing or completing manually, automatically, and/or a combination of thereof. The term “method” refers to manners, means, techniques, and procedures for accomplishing any task including, but not limited to, those manners, means, techniques, and procedures either known to the person skilled in the art or readily developed from existing manners, means, techniques and procedures by practitioners of the art to which the present disclosure belongs. The persons skilled in the art will envision many other possible variations within the scope of the present system and methods described herein.

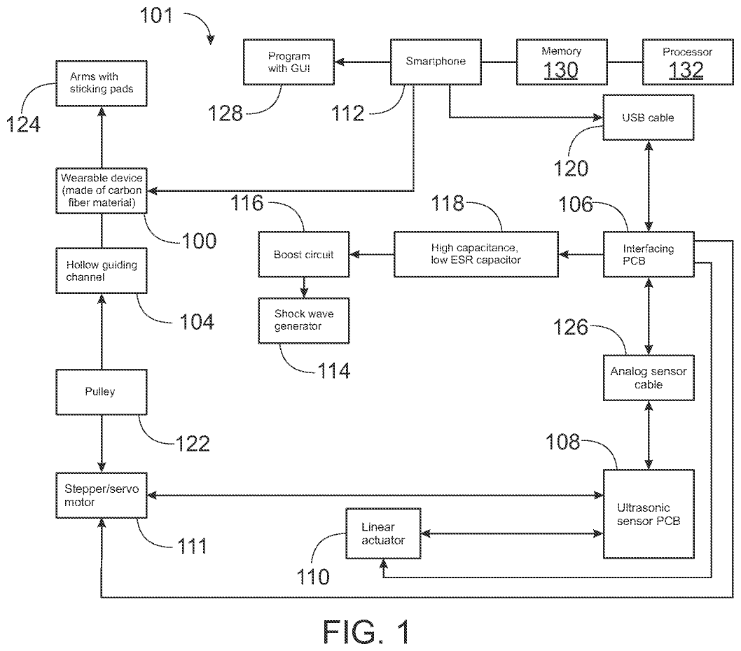

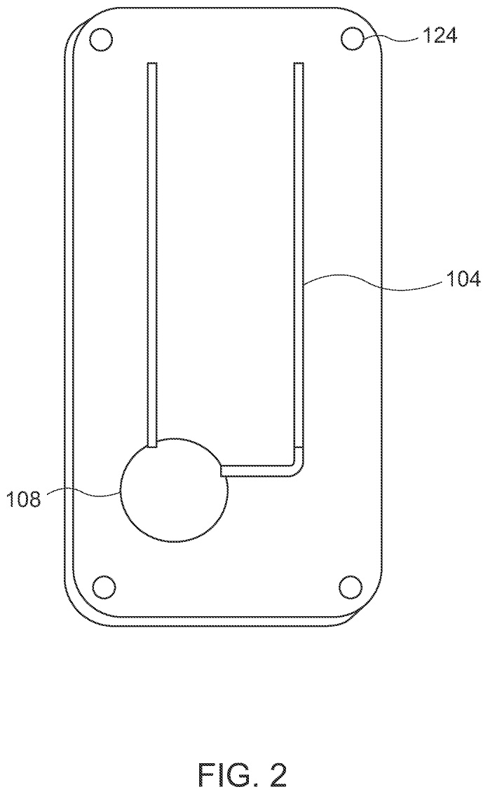

FIG. 1 illustrates a block diagram 101 of the connections between the various components of one exemplary embodiment of the presently disclosed device 100 (assembled views shown and explained in conjunction with FIGS. 2-4) for generating extracorporeal shock waves in a thoracic region of a user. The wearable device 100 includes a guiding channel 104, a circuit board (PCB) 106, a cardiac sensor in the form of an ultrasonic sensor 108, a micro linear actuator 110, a servo motor 111, and a processor 132. In some embodiments, the wearable device 100 acts as a gadget for the handheld computing device. Examples of the gadget include but are not limited to a casing, a cover, a housing, or an electrical housing. In some embodiments, the wearable device 100, or at least the housing/body thereof, is made of a carbon fiber material. Examples of handheld computing device 112 include but are not limited to a computing device, smartphone, a mobile device, a phablet, a tablet, etc.

The guiding channel 104 is placed into the body of the wearable device 100. The PCB 106 is connected to one or more pressure sensors, a shock wave transducer unit 114 and a processor 132. The pressure sensors are configured to measure the proximity of a shock wave transducer to the user's skin. The pressure sensors may include any instruments or devices that translate the magnitude of a physical pressure being exerted onto the sensor into an output signal that can be used to establish a quantitative value for the pressure. Pressure sensors may include but are not limited to potentiometric pressure sensors, inductive pressure sensor, capacitive pressure sensors, piezoelectric pressure sensors, strain gauge pressure sensors, variable reluctance pressure sensors, aneroid barometer sensors, Manometer Sensors, Bourdon Tube Pressure Sensors, Vacuum Pressure Sensors, Sealed Pressure Sensors.

The shock wave transducer unit 114 is configured to generate extracorporeal shock waves and is configured to be placed on the skin of the user with a sticking force chosen to optimize a shock wave therapy. The processor 132 is configured to execute a plurality of instructions, wherein the processor 132 is configured to transmit instructions for generating extracorporeal shock waves to the shock wave transducer unit.

The guiding channel 104 is configured to position the soundwave transducer unit within an area in the thoracic region of the user. In an embodiment, the PCB 106 further includes cardias sensor in the form of an ultrasonic sensor 108 which is connected to the circuit board (PCB) 106 via an analog sensor cable 126. In an embodiment, the ultrasonic sensor 108 includes a MEMS ultrasonic sensor. In an embodiment, the ultrasonic sensor 108 is a piezoelectric ultrasonic sensor.

The shock wave transducer unit 114 is attached to the PCB 106 via a spring-based mechanism to generate extracorporeal shock waves. The pressure sensors measure the proximity of the shock wave transducer unit and/or the ultrasonic sensor 108 to the skin. The ultrasonic sensor 108 is configured to detect different regions of the user's heart and determine a location on the chest where the shock wave transducer unit 114 is placed. The micro linear actuator 110 is attached to the ultrasonic sensor 108 to acquire Pulse Width Modulation (PWM) control from the interfacing circuit board 106. The micro linear actuator 110 is configured to place the ultrasonic sensor 108 on the skin of a user to obtain analog data with a sticking force chosen to minimize an error resulting from pressing the skin with the ultrasonic sensor 108.

The servo motor 111 is attached to the micro linear actuator 110 to acquire the PWM control from the interfacing circuit board 106 via a bidirectional PWM driver on the interfacing circuit board 106. In some embodiments, the micro linear actuator 110 includes a static side and a moving stroke and is attached to the servo motor 111 from the static side, and the ultrasonic sensor 108 is attached to the moving stroke of the micro linear actuator 110. In some embodiments, the pressure sensors, the shock wave transducer 114, and the piezoelectric ultrasonic sensor are soldered to a slim printed circuit board (PCB).

In some embodiments, the servo motor 111 moves in a plurality of channels created in the body of the wearable device 100. In some embodiments, functions of the servo motor 111 may be performed by a stepper motor. The servo motor 111 is powered and controlled by the interfacing circuit board (PCB) 106. In some embodiments, the servo motor 111 is attached to the micro linear actuator 110 to acquire the PWM control from the interfacing circuit board 106 via a bidirectional PWM driver placed on the interfacing circuit board 106. In some embodiments, the hollow guiding channel 104 is built into the body of the wearable device 100 to guide and restrict the movement of the servo motor 111.

The processor 132 is configured to execute a plurality of instructions stored in a memory 130 of the handheld computing device 112. The memory 130 may be a non-volatile memory or a volatile memory.

The processor 132 is configured to estimate the cardiac function from the cardiac sensor 108 measurements and transmit instructions for generating extracorporeal shock waves to the shock wave transducer unit 114. According to an embodiment herein, the processor 132 is configured to: identify regions of the user's heart; control an ultrasound therapy; adjust to a plurality of soundwave properties of a cardiovascular tissue to optimize penetration and efficacy of the ultrasound therapy; scan the user's heart, and detect one or more heart diseases; determine an effective ultrasound therapy for the user in one or more regions of the user's body; move the one or more shock wave generators to the one or more regions and execute the ultrasound therapy in said one or more regions; scan the heart by moving the ultrasound sensor and/or an electronic stethoscope across different regions of the user's thoracic region and collecting data therefrom; adjust ultrasound therapy based on the user's physiological and/or demographic characteristics; compare the cardiac health of the user in the one or more regions to determine an efficacy of the ultrasound therapy over time; and update the ultrasound therapy based on the observed efficacy of the ultrasound therapy over time.

In some embodiments, the interfacing circuit board 106 includes an analog-to-digital converter (ADC) to convert the analog data into digital data, a micro-controlling unit with a power and data transmission port, one or more large bandwidth operational amplifiers circuits, a plurality of digital buffers, at least two signal mixers for precise doppler calculation, a plurality of filters suitable for an operating range of the piezoelectric ultrasonic sensor 108, a plurality of bidirectional drivers for the micro linear actuator 110 and the servo motor 111, a plurality of headers and a plurality of PWM lines to provide the power to the micro linear actuator 110 and the servo motor 111. In some embodiments, the interfacing circuit board (PCB) is connected to the handheld computing device 112 via a power and data transmission cable 120 with data and power lines. The power and data transmission cable 120 receives power from the handheld computing device 112.

In some embodiments, the shock wave transducer 114 obtains power feed from a boost circuit 116. In some embodiments, the boost circuit 116 utilizes an accumulated charge on high capacitance and a low equivalent series resistance (ESR) capacitor 118. The accumulated charge is acquired from the handheld computing device 112 via the power and data transmission cable 120 during idle time.

In some embodiments, wearable device 100 includes a pulley 122 attached to the servo motor 111 to move the shockwave transducer unit inside the hollow guiding channel 104 in the body of the wearable device. In some embodiments, the wearable device 100 includes a plurality of arms with fixation pads 124 are attached to the bottom of the case 102 allowing the gadget or device 100 to be attached to the user's skin.

In some embodiments, the wearable device 100 is powered by the handheld computing device 112 or may obtain power from an external battery that can supply electrical power to the PCB 106.

Since impairment of arterial endothelial function may indicate the start of cardiovascular disease, the brachial artery is measured for diameter before and after several minutes of either vasoconstriction or vasorelaxation, and when an ultrasonic signal is transmitted and retrieved at the PCB of the ultrasonic sensor 108, a Doppler shift occurs between both sent and received pulse which indicates the flow speed of blood, and any change in that speed reflects a corresponding change in the artery diameter. The delay between pulse application and detection can be measured using a signal mixer, where the RMS value of the resulting multiplication of signals indicates the shift between the 2 signals, while an integrator circuit can be used to measure the change in Doppler shift, where a steady flow must have a linear integration result, while the degree of un-linearity measures the Doppler shift since this un-linearity is proportional to the change in flow speed.

To avoid any measurement error caused by the weight of the measurement device, a hollow case made from carbon fibers similar to that of the handheld computing device can be used to create a hollow space around the sensor to guarantee there is no extra weight from the wearable device on the skin between the sensor and artery, and thus guarantee precise results, so the pressure applied to the skin by the wearable device 100 is exerted at points far from the artery and has no effect on the artery diameter or shape.

FIG. 2 illustrates a bottom view of the wearable device, in accordance with one embodiment of the present disclosure. FIG. 2 is explained in conjunction with FIG. 1. The arms with fixation pads 124 are configured to be placed on the body of the patient. The hollow guiding channel 104 is placed into the body of the wearable device 100 to guide the shock wave transducer unit via movement of the stepper motor or servo motor 111 and the linear actuator 110 and PCB of the ultrasonic sensor 108 attached to it. The PCB of the ultrasonic sensor 108 is attached to the stepper motor 111 via the linear actuator 110.

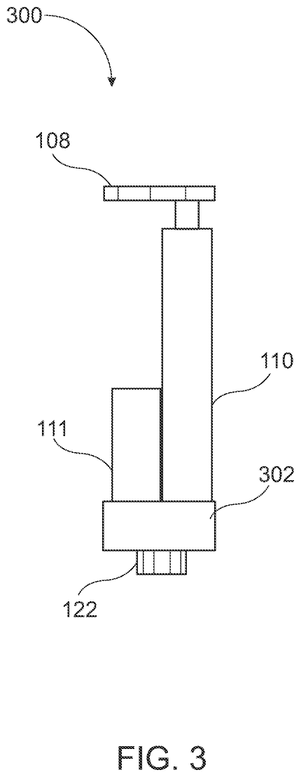

FIG. 3 illustrates a side view 300 of the wearable device, in accordance with one embodiment of the present disclosure. FIG. 3 is explained in conjunction with FIG. 1. A cardiac sensor in the form of a piezoelectric ultrasonic transceiver 108, a shock wave transducer unit comprising a highly directional extracorporeal ultrasonic shock wave transducer, and one or more pressure sensors are soldered to a slim PCB (preferably 0.4 mm or less PCB). In some embodiments, wearable device 100 includes a driving motor 302 for the linear actuator. The pulley 122 is attached to the stepper/servo motor 111 and is meant to move only in the hollow guiding channel 104 in the body of the wearable device 100.

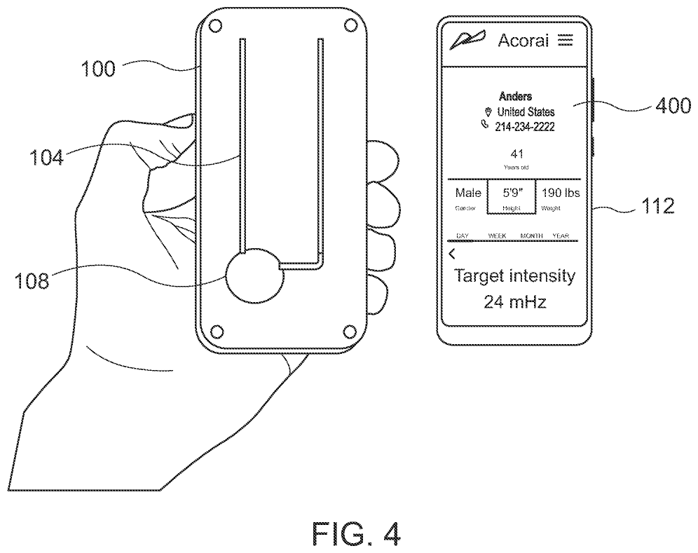

FIG. 4 illustrates a monitoring application 400 installed within the handheld computing device 112, in accordance with one embodiment of the present disclosure. FIG. 4 is explained in conjunction with FIG. 1. The monitoring application 400 may be based on one or more operating systems comprising Android®, and iOS®. The wearable device 100 requires the user to register on the monitoring application 400 installed or configured within the handheld computing device 112. Memory 130 is configured to register the user over the monitoring application 400 by receiving one or more credentials from the user for providing access to the monitoring application 400. Examples of the credentials including but not limited to a user name, password, age, gender, phone number, email address, location, etc. In some embodiments, the monitoring application 400 is commercialized as a software application or a mobile application, or a web application for cardiac health assessment. A user may include a patient, a patient using the monitoring application using the handheld computing device 112 such as those included in this invention, or such a handheld computing device 112 itself. In some embodiments, the monitoring application 400 is a combination of a software program with a graphical user interface (GUI) 128 (shown in FIG. 1) which is running on the handheld computing device 112 to present resulting data such as name, location, age, gender, height, weight, periodical target intensity, etc. and allow the user to do suitable adjustments based on the resulting data. The resulting data is obtained by one or more ultrasonic sensors 108 configured with the wearable device 100.

According to an embodiment herein, processor 132 processes the captured/obtained data and transmits it to an external computing device or as a server for further processing over a network. The processed data related to the heart's health of the user is presented on the monitoring application 400. The network may be a wired or a wireless network, and the examples may include but are not limited to the Internet, Wireless Local Area Network (WLAN), Wi-Fi, Long Term Evolution (LTE), Worldwide Interoperability for Microwave Access (WIMAX), and General Packet Radio Service (GPRS).

The monitoring application 400 enables the user to continuously monitor the probability of heart failure and helps in the treatment of heart failure with preserved ejection fraction (HFpEF). Typically, HFpEF occurs when the lower left chamber (left ventricle) is not able to fill properly with blood during the diastolic (filling) phase. The amount of blood pumped out to the body is less than normal. It is also called diastolic heart failure. Further, the monitoring application 400 utilizes machine learning for automatic positioning and determining the intensity of ultrasonic transducers.

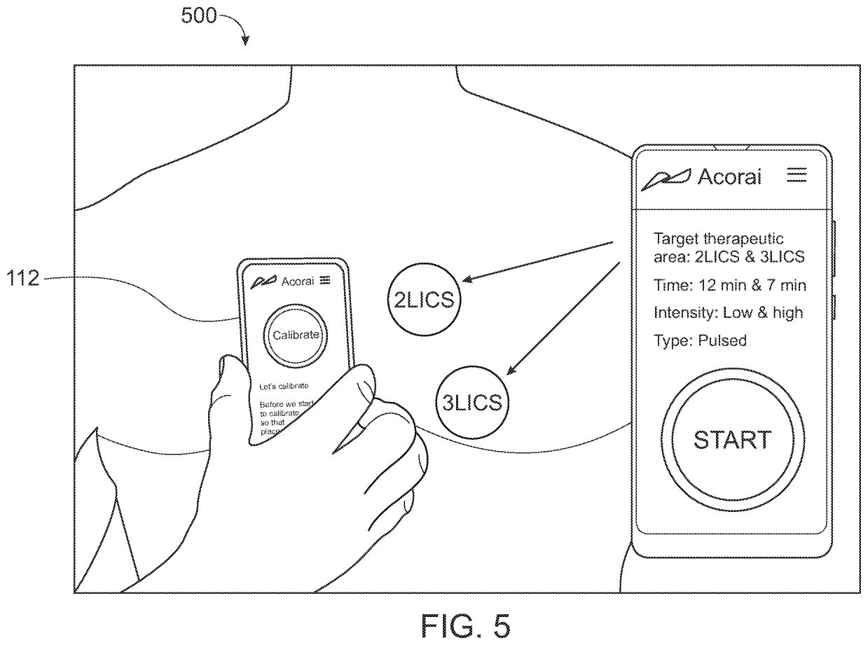

FIG. 5 illustrates a perspective view 500 of the handheld computing device placed against the body or chest of a user, in accordance with at least one embodiment. FIG. 5 is explained in conjunction with FIG. 4. The monitoring application 400 directs the user through the GUI to start the measurement of cardiac function. Then the user places the handheld computing device against his/her chest as shown in FIG. 5. The handheld computing device 112 may have a shape adapted to fit firmly on the user's chest. The shape of the handheld computing device 112 is bend or curved so that it perfectly fits on the patient's chest.

In some embodiments, the cardiac sensor 108 is combined with the one or more proximity sensors, such as pressure sensors, that allow the linear actuator to place the shock wave transducer unit and/or the cardiac sensor right on the skin with minimum and fixed sticking force to minimize the error resulting from pressing the skin with a probe. According to an embodiment herein, wearable device 100 utilizes a closed-loop control using a digital PID algorithm to ensure that the applied force to the skin doesn't cause additional errors to the measurement process. Further, wearable device 100 utilizes the closed-loop control using the digital PID algorithm to ensure that the position of the sensor is optimized automatically to make sure that the applied measurement cannot be optimized further.

The signal coming from the pressure sensors is digitized and sent to the MCU to use its full processing power, and use Fourier libraries or other digital processing libraries to determine the Doppler shift accurately and in a real-time manner with minimum additional hardware or cost. The signal processing of the array signals in a preferred enablement will include treating the signals as complex using the quadrature representation, which also should include Hilbert transforms to generate analytic signals necessary for phased-array optimal processing and control, and for matched filtering. Finally, the highly directional extracorporeal ultrasonic shock wave transducer can be used with high accuracy due to the precision ultrasonic transceiver, so the wearable device 100 can also provide therapy, and not just detection or measurement. Because the accuracy of Doppler shift for a wave is proportional to the frequency of the wave, optimum frequency is around 8 MHZ, while standard Doppler calculation is by estimating pulse wave velocity, by dividing the distance between the sensor by the pulse transit time.

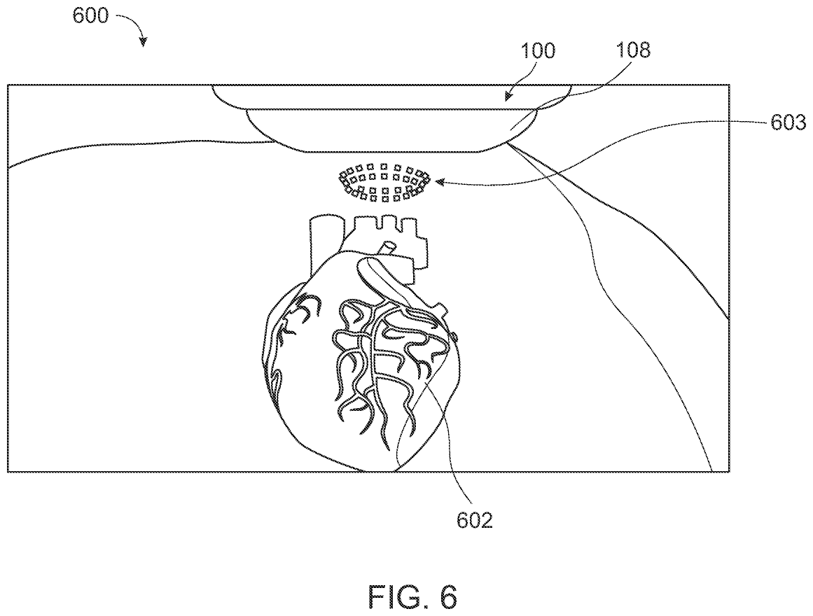

In some embodiments, the cardiac sensor 108 uses an integrator as a part of ultrasonic calculator circuits is an ideal solution where sampling of the direct output waveform can introduce many problems since the output waveform doesn't have an exact wave shape, while an integrator allows measurement of the changes by determining the nonlinearity of the resulting waveform from the integrator. Finally, the use of analog filters is critical to make sure that any noise from external sources is neglected so that the input of the integrator is guaranteed to be from the ultrasonic reading rather than ambient EM waves at the integrator input. FIG. 6 illustrates a perspective view 600 of interactions between the ultrasonic sensor 108 and heart 602 of the user, in accordance with at least one embodiment. In operation, the interfacing circuit board (PCB) 106 retrieves audio signal from the PCB of the ultrasonic sensor 108. The audio signal informs the micro-controlling unit (MCU) of the interfacing circuit board (PCB) 106 about the optimum X-Y position to place the ultrasonic sensor 108 at the automated control uses audio level as means of choosing the optimized position of the sensor, where the feedback resulting from audio recognition of artery pulses helps the MCU to recognize the position that allows maximum audio retrieval of the pulses and thus producing PWM power to control the servo/stepper motor based on the feedback from the audio signal. The process of sticking the ultrasonic sensor 108 to the skin is provided by means of the positioning mechanism using a linear actuator attached to the stepper/servo motor, positioning process is done with the help of pressure sensors designed to limit the sticking force to a fixed value to reduce errors resulting from extra pressure on the skin that may affect the artery diameter or shape. The interfacing circuit board (PCB) 106 generates PWM power pulses to control the linear actuator based on the measurement coming from the proximity sensors. Optimizing shock wave therapy may include placing the shock wave transducer unit on the skin of a user with a sticking force chosen to minimize an error resulting from pressing the skin with the shock wave transducer.

In a further embodiment, the wearable device may be individually calibrated to each user so that ultrasound therapy parameters is determined based on a number of factors, including but not limited to the one or more diseases that are being targeted, demographic information of the user, health information of the user, ultrasound wave 603 penetrability of the user's skin, tissue, bone and/or organs, the environment that the ultrasound therapy will be performed in, the time of day that the ultrasound therapy will be performed in, and/or the risk of adverse health events occurring for the user. Individual calibration of the wearable device may further include adjusting the size of the device to fit different body sizes and/or different physiological properties of different genders.

The cardiac sensor and the shock wave transducer can be separated entities or they can be incorporated in the shock wave transducer unit. The cardiac sensor may comprise a high volt ultrasonic transceiver that can generate a highly directional shock wave which is delivered to the user via the shock wave transducer. A handheld computing device power may provide a volt limited to 5V level only, but a high efficiency boost circuit can be added to the PCB 106, and if the boost circuit makes use of a low ESR, high capacitance capacitor, i.e., a supercapacitor that allows a boost circuit to provide high volt, high current for a very short period i.e. 1 or 2 milliseconds, i.e. enough for the shock wave. Since the shock wave transducer doesn't need to be precisely stuck to the skin without pressure, the shock wave transducer can be an independent PCB statically attached to the bottom of the hollow case in a manner that doesn't conflict with the movement trajectory of the cardiac sensor 108, a simple spring mechanism can be used to make sure that it is stuck to the skin with enough pressure.

The interfacing circuit board (PCB) 106 filters and mixes the signal used to transmit ultrasonic pulse with the signal retrieved during ultrasonic sensing and integrate the result and send sampled/digitized results to the handheld computing device CPU via the power and data transmission cable so that the handheld computing device CPU, while a handheld computing device can use cheap and existing digital signal processing libraries i.e. Fast Fourier transform libraries for Android®, IOS®, Windows® and Linux® to minimize the programming development cost to calculate the Doppler shift based on the un-linearity In the integrated signal, and thus determine the flow speed and changes.

The interfacing circuit board (PCB) 106 collects data from the ultrasonic sensor 108 and sends it back to the handheld computing device. Finally, the handheld computing device retrieves back the ultrasonic data in a digitized form and uses this data for analyzing Doppler shift to determine the flow speed of blood and thus the arterial changes, presenting the resulting data in a readable, graphic, or audio form i.e., GUI. Thus, the present wearable device 100 harnesses the thermal and nonthermal effects of high-intensity focused ultrasound (HIFU) and extracorporeal shock waves to treat heart diseases.

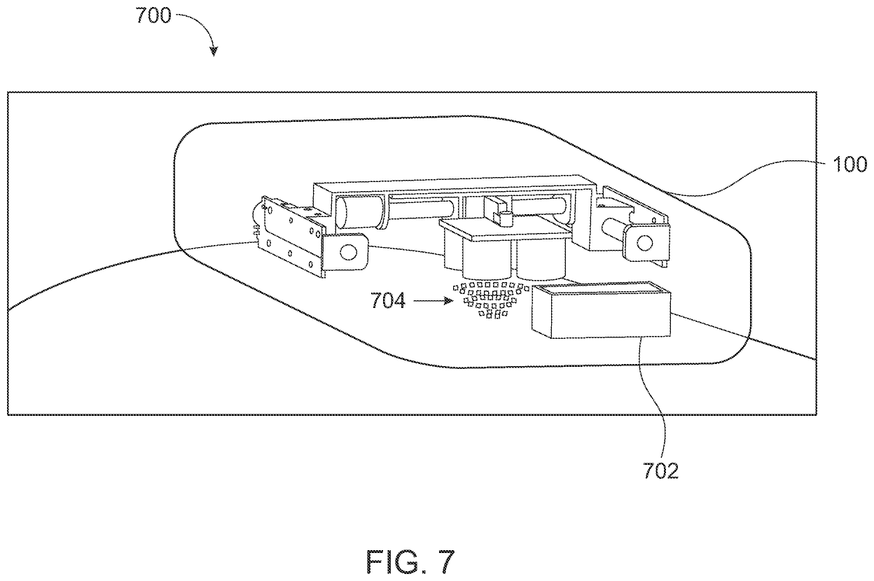

FIG. 7 illustrates a first exploded view 700 of the placement of the wearable device 100 against the chest of the user, in accordance with at least one embodiment. According to an embodiment herein, the wearable device 100 includes a container 702 to store acoustic impedance matching liquid.

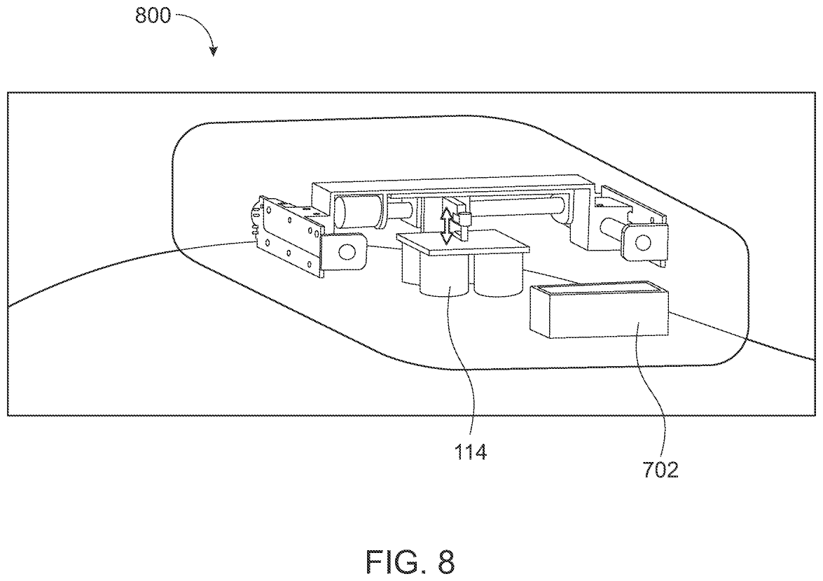

FIG. 8 illustrates a second exploded view 800 of the placement of the device against the chest of the user, in accordance with at least one embodiment. The pulsation frequency and strength of the shock waves generated by the shock wave transducer unit 114 are directed at suppressing hypertrophic cardiomyopathy and/or myocardial interstitial fibrosis.

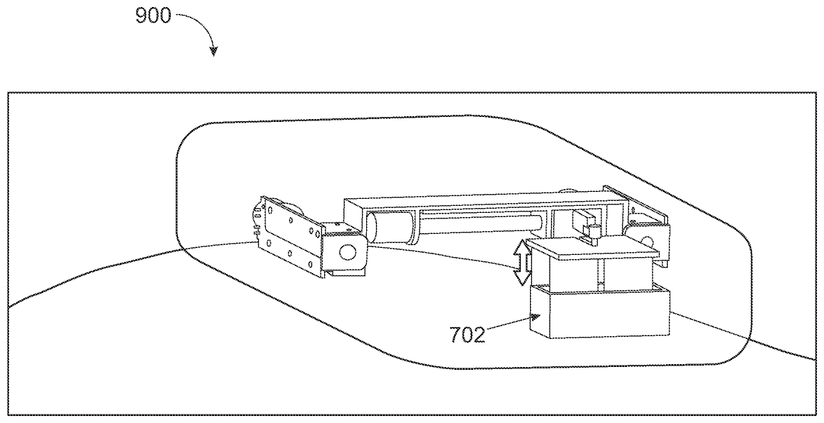

FIG. 9 illustrates a third exploded view 900 of the placement of the wearable device against the chest of the user, in accordance with at least one embodiment. In an embodiment, the shock wave transducer unit 114 includes an electrohydraulic source, and/or a piezoelectric source, and/or an electromagnetic source with a flat coil, and/or an electromagnetic source with a cylindrical coil.

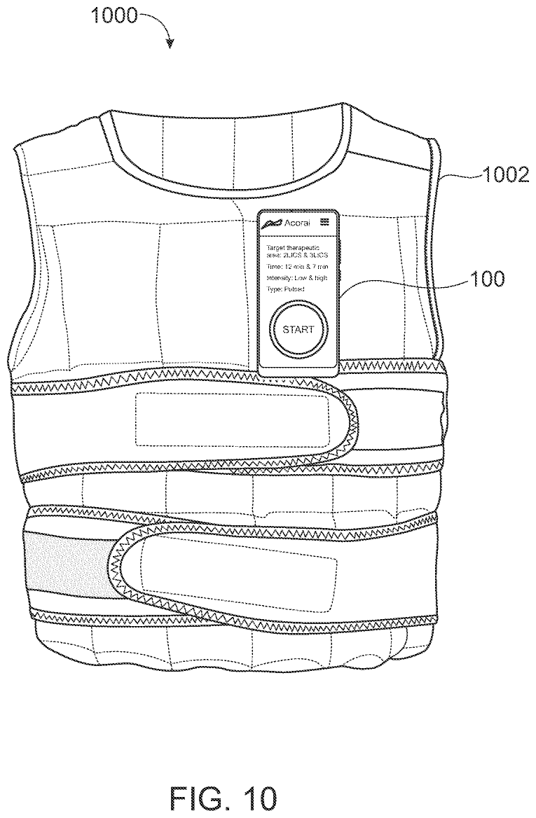

FIG. 10 illustrates a perspective view 1000 of placing the wearable device along with the handheld computing device into a pocket of a vest 1002, in accordance with at least one embodiment. The wearable device 100 can be put into the pocket of the vest 1002 to enable a controlled and stable placement of the wearable device 100 during ultrasound therapy. In an embodiment, the vest 1002 is configured to ensure a good fit with the user's chest. The vest 1002 comes in different sizes to ensure a good fit across different patient populations. The different sizes of the vest 1002 facilitate the wearable device 100 to be used in the region of a user across different user populations. Further, the vest 1002 can be adjusted to increase pressure and/or improve contact of sensors and/or transducers with the patient's skin.

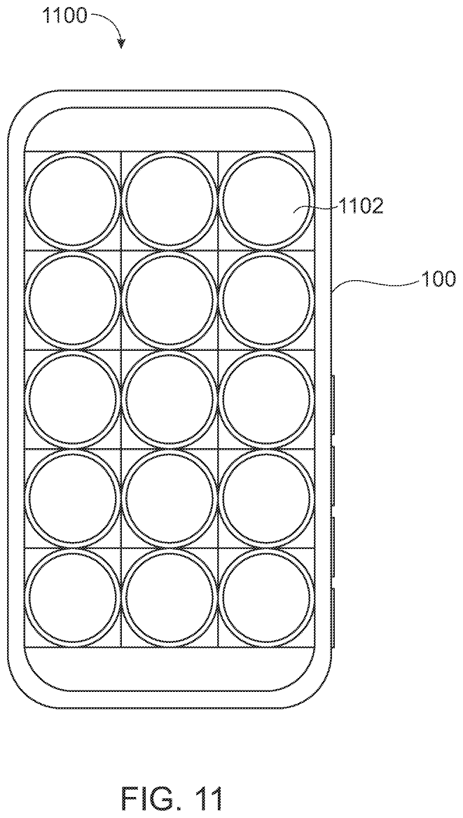

FIG. 11 illustrates a perspective view 1100 of a matrix or array of ultrasound transducers or transducer units 1102, in accordance with at least one embodiment. In another embodiment, the wearable device 100 of the present wearable device 100 includes a matrix of ultrasound transducers 1102 instead of the linear actuators and a guiding channel. According to an embodiment herein, different transducers 1102 are activated depending on which region of the heart needs treatment. In an embodiment, the shock wave transducers are activated in accordance with the region of the heart that is to be analyzed and/or treated. The shock wave transducers can be adjusted electronically and/or mechanically to ensure a good fit with the user's skin.

In one embodiment, the array of transducer units can be configured to generate ultrasound signals or pulsations for ultrasound therapy and in addition be configured for providing an electrical signal in response to an incoming ultrasound signal or pulse.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

### Items

1. A wearable device for generating extracorporeal shock waves in a thoracic region of a user, the wearable device comprising:
   a shock wave transducer unit to generate extracorporeal shock waves and configured to be placed on the skin of the user to apply shock wave therapy; and
   at least one proximity sensor to measure the proximity of the shock wave transducer unit; and
wherein the one or more shock wave transducer units are arranged in an array, or
wherein the wearable device further comprises a positioning mechanism configured to controllably position the shock wave transducer unit;
wherein the device is configured to receive cardiac health information, and transmit information to the shock wave transducer unit for generating extracorporeal shock waves.
2. The wearable device of item 1, comprising a cardiac sensor.
3. The wearable device of item 2, wherein the cardiac sensor is configured for non-invasively acquiring cardiac data of the user.
4. The wearable device of item 2, wherein the cardiac sensor is an invasive cardiac sensor.
5. The wearable device of any of the preceding items, wherein the cardiac health information comprises cardiac sensor data and/or self-reported cardiac health data.
6. The wearable device of any of the preceding items, wherein the positioning mechanism comprises a guiding channel for guiding the shock wave transducer unit and/or the cardiac sensor in a plane defined by a bottom surface of the device towards the skin of the user.
7. The wearable device of any of the preceding items, further comprising a circuit board, such as PCB, for connecting the shock wave transducer unit, the proximity sensor(s), the cardiac sensor(s), the positioning mechanism and the processor.
8. The wearable device of any of the preceding items, wherein the proximity sensor is a pressure sensor for measuring the pressure that the shock wave transducer applies on the user's skin.
9. The wearable device of any of the preceding items, wherein the cardiac sensor comprises an ultrasonic sensor for measuring a cardiac function of the heart of the user.
10. The wearable device of any of the preceding items, wherein the cardiac sensor comprises an electronic stethoscope.
11. The wearable device of any of the preceding items, further comprising a photoplethysmography (PPG) sensor.
12. The wearable device of any of the preceding items, further comprising an electrocardiogramansor.
13. The wearable device of any of the preceding items, further comprising an inertial measurement unit (IMU) sensor, such that the device is configured to determine whether the device has been correctly positioned.
14. The wearable device of any of the preceding items, wherein the positioning mechanism comprises a spring-based mechanism for engaging with the shock wave transducer unit.
15. The wearable device of any of the preceding items, wherein the positioning mechanism comprises a motor for engaging with the shock wave transducer unit.
16. The wearable device of item 15, wherein the positioning mechanism comprises a pulley engaging with the motor.
17. The wearable device of item 16, wherein the device is configured such that the motor and the pulley move controllably to position the shock wave transducer unit and/or the cardiac sensor(s).
18. The wearable device of any of the preceding items, wherein the positioning mechanism comprises a micro linear actuator.
19. The wearable device of item 18, wherein the micro linear actuator comprises a driving side and a driven side for engaging with a motor from the driving side, and for engaging with the shock wave unit from the driven side.
20. The wearable device of any of the preceding items, configured such that the positioning mechanism provides Pulse Width Modulation (PWM) control of the shock wave transducer unit.
21. The wearable device of item 20, further comprising a bidirectional PWM driver for the PWM control.
22. The wearable device of any of the preceding items, further comprising a housing for accommodating at least a part of the shock wave transducer unit, the proximity sensor, the positioning mechanism and the processor, wherein the housing is made of a carbon fiber material.
23. The wearable device of any of the preceding items, wherein the device is configured to receive analog data from the user.
24. The wearable device of any of the preceding items, wherein the device is configured to analyze a cardiac function of the heart of the user and determine a location on the chest of the user where the shock wave transducer unit is positioned based on sensor data of the shock wave transducer unit.
25. The wearable device of any of the preceding items, wherein the PCB is connected to one or more of the following:
   an analog-to-digital converter (ADC) for converting analog ultrasonic data into digital data,
   a micro-controlling unit with a power and data transmission port,
   one or more large bandwidth operational amplifiers circuits,
   a plurality of digital buffers,
   at least two signal mixers for precise doppler calculation,
   a plurality of filters suitable for an operating range of a piezoelectric ultrasonic sensor,
   a plurality of bidirectional drivers for a micro linear actuator and a servo motor,
   a plurality of headers and a plurality of PWM lines to provide the power to the micro linear actuator and the servo motor.
26. The wearable device of any of the preceding items, wherein the interfacing circuit board (PCB) is connected to a mobile computing device via a cable with data and power lines, wherein the cable receives power from the mobile computing device.
27. The wearable device of any of the preceding items, comprising a boost circuit for providing power feed to the shock wave transducer unit.
28. The wearable device of item 27, wherein the boost circuit comprises a low equivalent series resistance (ESR) capacitor and utilizes an accumulated charge on high capacitance.
29. The wearable device of any of the preceding items, comprising a plurality of fixation pads at the bottom surface of the wearable device for allowing the wearable device to be attached to the user's skin.
30. The wearable device of any of the preceding items, comprising a container having acoustic impedance matching liquid.
31. The wearable device of any of the preceding items, further comprising a memory and/or a server for providing instructions to the device.
32. The wearable device of any of the preceding items, wherein the information comprises information on ultrasound therapy parameters including but not limited to information on location, frequency, spatial average temporal average, duty cycle and/or duration of therapy.
33. The wearable device of any of the preceding items, comprising a display screen.
34. The wearable device of any of the preceding items, comprising a battery.
35. The wearable device of any of the preceding items, configured to be individually calibrated to the user.
36. The wearable device of any of the preceding items, configured to scan the user's heart, identify one or more regions of the user's heart and detect one or more heart diseases.
37. The wearable device of any of the preceding items, configured to determine an effective ultrasound therapy for the user in one or more regions of the user's thoracic region.
38. The wearable device of any of the preceding items, configured to move the shock wave transducer unit to the one or more regions and execute the ultrasound therapy in said one or more regions.
39. The wearable device of any of the preceding items, configured to scan the heart by moving the ultrasound sensor and/or an electronic stethoscope across different regions of the user's thoracic region and collecting data therefrom.
40. The wearable device of any of the preceding items, configured to compare the cardiac health of the user in the one or more regions to determine an efficacy of the ultrasound therapy over time.
41. The wearable device of any of the preceding items, configured to update the ultrasound therapy based on the observed efficacy of the ultrasound therapy over time.
42. The wearable device of any of the preceding items, configured to create a map of the user's heart, wherein the map is stored in the memory.
43. The wearable device of any of the preceding items, wherein the wearable device is configured to collect information on the user's health condition through a questionnaire and/or patient health database.
44. The wearable device of any of the preceding items, wherein the wearable device is connected to a vest.
45. The wearable device of any of the preceding items, wherein the wearable device comprises a manual pressure adjustment knob configured to move the shockwave transducer unit up and down.
46. The wearable device of any of the preceding items, wherein the wearable device receives power through a cable from an external electric power system.

1. A wearable device for generating extracorporeal shock waves in a thoracic region of a user, the wearable device comprising:
at least one array of shock wave transducer units to generate extracorporeal shock waves and configured to be placed on the skin of the user to apply shock wave therapy; and
at least one proximity sensor to measure the proximity of the one or more shock wave transducer units;
wherein the device is configured to receive cardiac health information, and transmit therapy information to the array of shock wave transducer units for generating extracorporeal shock waves.

2. The wearable device of claim 1, comprising a cardiac sensor configured for generating the cardiac health information.

3. The wearable device of claim 1, wherein the cardiac health information comprises cardiac sensor data and/or self-reported cardiac health data.

4. The wearable device of claim 1, wherein the proximity sensor comprises a pressure sensor configured for measuring the pressure that the array of shock wave transducer units applies on the user's skin.

5. The wearable device of claim 1, wherein the cardiac sensor comprises an ultrasonic sensor or an electronic stethoscope for measuring a cardiac function of the heart of the user.

6. The wearable device of claim 2, wherein the cardiac sensor is the array of shock wave transducer units.

7. The wearable device of claim 1, configured to scan the heart by collecting data using the cardiac sensor across different regions of the user's thoracic region and wherein the device is configured to analyze a cardiac function of the heart of the user and determine a location on the chest of the user where the array of shock wave transducer units shall transmit extracorporeal shock waves based on said cardiac sensor data.

8. The wearable device of claim 1, comprising a plurality of fixation pads at a bottom surface of the wearable device for allowing the wearable device to be attached to the user's skin.

9. The wearable device of claim 1, comprising a container for containing acoustic impedance matching liquid for use during shock wave therapy.

10. The wearable device of claim 1, wherein the transducer units are controllable independently for controlling the direction and/or the focal length of the generated extracorporeal shock waves.

11. The wearable device of claim 1, wherein the array is one-dimensional or two-dimensional.

12. The wearable device of claim 1, wherein the at least one array of shock wave transducer units is configured to generate electric signals as responses to reflected ultrasound waves from the heart.

13. The wearable device of claim 1, wherein the wearable device comprises an acoustic transducer, like a microphone, like an accelerophone, for detecting sound waves below 20 kHz or below 15 kHz, or the wearable device is configured to detect cavitation using the shock wave transducer units.

14. The wearable device of claim 1, wherein the wearable device is configured to emit extracorporeal shockwaves to induce a regenerative cardiac therapy.

15. The wearable device of claim 1, wherein the wearable device comprises one or more cardiac rhythm detection sensors.

16. The wearable device of claim 1, wherein the wearable device comprises one or more breath detection sensors.

17. The kit comprising a vest for fitting a user's chest and the wearable device of claim 1, wherein the vest is configured to receive and attach the wearable device.
